(19) United States
(12) Patent Application Publication
(10) Pub. No.: US 2013/0197463 A1
(43) Pub. Date: Aug. 1, 2013

(54) ABSORBENT DISPOSABLE INCONTINENCE DIAPER HAVING SIDE SECTIONS

(71) Applicant: Krzysztof D. Malowaniec, Heidenheim (DE)

(72) Inventor: Krzysztof D. Malowaniec, Heidenheim (DE)

(73) Assignee: Paul Hartmann AG, Heidenheim (DE)

(21) Appl. No.: 13/809,076

(22) PCT Filed: Jul. 5, 2011
(86) PCT No.: PCT/EP2011/003338

§ 371 Date: Jan. 29, 2013

(30) Foreign Application Priority Data
Jul. 9, 2010 (DE) 10 2010 026 643.4

(51) Int. Cl. A61F 13/56

(52) U.S. Cl. CPC A61F 13/5644
USPC 604/386

(57) ABSTRACT

An open-type absorbent disposable incontinence diaper, includes a main part which includes an absorbent element, a front region having front lateral longitudinal edges, a rear region having rear lateral longitudinal edges, and a crotch region arranged between the front and rear regions. The diaper also includes rear side sections joined to the rear region on both sides and front side sections joined to the front region on both sides, said sections extending in the transverse direction beyond the lateral front and rear longitudinal edges of the main part. For forming rear side-section leg opening regions, a lower edge of a rear side section is curve-shaped. A ration of a length of the lower edge to a width of the lower edge is 0.40-0.90 and a ratio of a longitudinal extent of an extreme point of a convex portion of the lower edge to a transverse extent of the extreme point is 0.15-0.80.

The invention relates to a disposable absorbent incontinence article of the open type, and therefore a disposable incontinence diaper for adults, having a main part, consisting of a front region, a rear region and a crotch region that is located in between in the longitudinal direction and comes to lie between the legs of a user, wherein the main part comprises an absorbent pad, and having separate side portions that are attached to the rear region and to the front region on both sides and which are provided separately from one another with closure means, extend in the transverse direction beyond lateral longitudinal edges of the main part and connect the front region and the rear region together in the put-on state of the article.

Incontinence articles of this kind are known and described, for example, in WO 2005/102241 A1. The side portions, which are sometimes also referred to as ears, are preferably attached directly to the main part, i.e. the chassis of the sanitary article, on both sides in a cut and place method. This manufacturing technology makes it possible to manufacture the side portions from a different raw material than the central main part of the sanitary article. For example, the side portions can be configured in an air-permeable manner, whereas the central main part can be formed in a substantially moisture-impermeable manner.

The most efficient and simplest, and also most cost-effective, shape of the side portions from a manufacturing point of view is the rectangular shape. This allows the materials forming the side portions to be transported during production in the form of an endless web of flat material from which the side portions are then severed transversely to the machine direction. There are virtually no offcuts in this case.

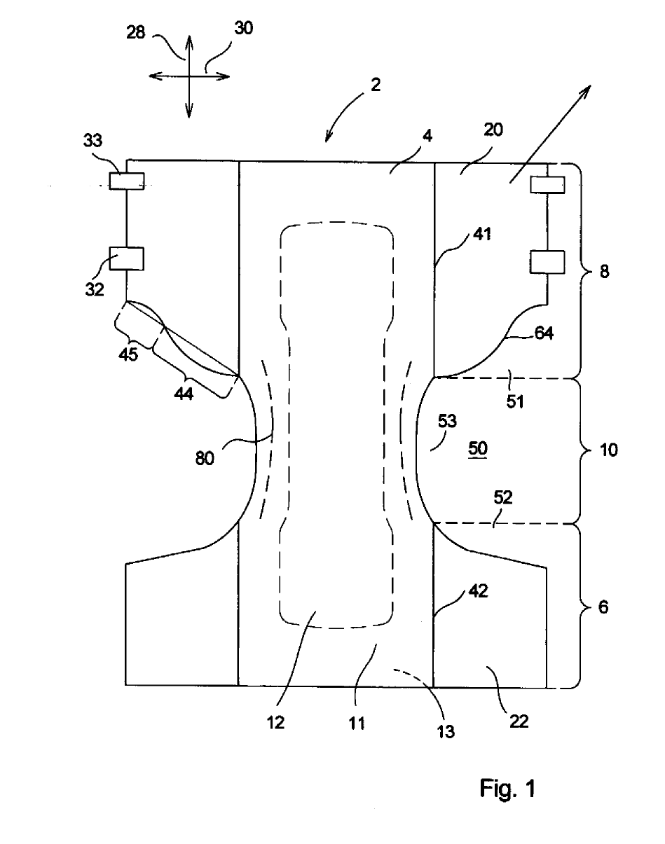

However, it has been shown that, particularly during the formation of the side portions in the otherwise advantageous rectangular form, when the sanitary article is being put on and while it is being worn, the problem sometimes occurs that the attached side portions can tear in the region of the lateral longitudinal edges of the main part. It has specifically been shown that, when users put on the sanitary article, they tend to exert a pull, obliquely to the transverse and longitudinal directions of the sanitary article, on the side portions that extend extremely far in the transverse and longitudinal directions in the case of incontinence articles for adults, this being indicated in FIG. 1 by way of an obliquely upwardly inclined arrow. In such cases, it is possible for side portions to tear along the lateral longitudinal edges of the main part, with the tear propagating from the transverse edge, facing the crotch region, of the side portion. Hitherto, attempts have been made to improve the attachment of such side portions to the main part of sanitary articles by an optimum joining pattern, as per WO 2004/017882 A2 and WO 02/17843 A2.

A further proposal known from the prior art is to provide the side portions with a reinforcement means which, as seen in the transverse direction, is formed in a narrower manner than each side portion and which is provided at least in a region bridging the lateral longitudinal edge of the main part, that is to say overlays both a lateral longitudinal edge region of the main part and also a part of the side portion in the transverse direction (DE102006050971A1).

The present invention is based on the object of solving the problem outlined above in a more effective manner, that is to say to create disposable absorbent incontinence diapers having two side portions that are joined and attached at the rear and preferably also at the front, in the case of which the tearing behavior of the rear side portions is significantly improved.

In the case of a disposable absorbent incontinence diaper of the type mentioned at the beginning, this object is achieved according to the invention in that a lower edge of the rear side portions is formed in a curved manner, in that the lower edge has a convex portion, in that the ratio of the length A of the lower edge of a rear side portion to the width B of the lower edge of a rear side portion is 0.4-0.9 and the ratio of the longitudinal extent E of an extreme point P of the convex portion to the transverse extent F thereof is 0.15-0.80, wherein A, B, E and F are determined as described further below by way of the description of the figures.

As will also be explained in more detail by way of the description of the figures, in the context of the present invention the extreme point P is understood to be a point on a convex portion of the lower edge of the rear side portions, said point being at a maximum distance from an imaginary straight line which is drawn through the end points of the lower edge of a rear side portion. These end points are the points at which the lower edge meets the inner or outer edge of the side portions.

Surprisingly, this geometry according to the invention of the rear side portions leads to improved stretching behavior of the side portions in use and thus ultimately to a much improved tearing behavior of the rear side portions, without the fit of the disposable incontinence diaper being impaired.

The convex portion of the lower edge of a rear side portion, the position of the extreme point, i.e., as it were, the coordinates of the maximum of the convex portion in combination with the ratio of the longitudinal and transverse extent of the lower edge of a rear side portion, allow an improved tearing behavior of the rear side portions. This can be explained by a comparatively high amount of work (in mNm) required up until breakage of the materials in a use-simulating side-portion tearing test. Thus, more energy is required in use to damage the material.

Preferably, the work which is required up until breakage of the material in a use-simulating side-portion tearing test, described below, is at least 1900 mNm, particularly at least 1950 mNm, more particularly at least 2000 mNm, but preferably less than 4000 mNm.

Preferably, the ratio of the length A of the lower edge of a rear side portion to the width B of the lower edge of a rear side portion is 0.50-0.80, particularly 0.55-0.75.

In a development of the invention, it is proposed that the ratio of the transverse extent F of the extreme point P to the transverse extent B of each lower edge of the rear side portion is 0.20-0.60, particularly 0.30-0.50.

More preferably, the ratio of the longitudinal extent E of the extreme point P to the length A of a lower edge of each rear side portion is 0.10-0.40, particularly 0.10-0.30.

Advantageously, the ratio of the longitudinal extent E of an extreme point P of the convex portion to the transverse extent F thereof is 0.20-0.60, particularly 0.20-0.50.

Preferably, the tear propagation resistance Fm of the material forming the rear side portions, measured and determined as average force Fm, as described in more detail below, is at least 4.0 N in the longitudinal direction of the diaper. Given contouring according to the invention of the side portions, this makes it possible to position the closure means very close to the lower edge of the rear side portions, that is to say at most 8.0 cm away therefrom. While it is not denied that the above-described risk of the side portions tearing along the lateral longitudinal edges of the main part decreases by moving the closure means away from the lower edge of the side portions, this would at the same time entail a considerable loss of comfort in putting the diaper having the side portions that extend very far in the longitudinal and transverse directions of the diaper onto a person with a precise fit. Putting the diaper on with a precise fit is very much easier to carry out with closure means positioned very close to the lower edge of the side portions, since as a result a pull can be exerted over virtually the length of the side portions via the closure means.

In order to produce a disposable incontinence diaper according to the invention, first of all rectangular material portions are preferably attached to both sides of the main part of the diaper and the side-portion leg-opening regions are formed only subsequently by a continuously or quasi-continuously guided severing process, particularly by cutting or punching, and so a continuous edge is formed. DE102009015041 shows this method using the example of a disposable incontinence diaper having concavely contoured side portions. The line of the severing process in this case takes in the rear side portion, the main part and the front side portion. The leg-opening regions are thus formed preferably exclusively by cutting or separating edges of the single, continuous or quasi-continuous severing process, this of course also implying that the disposable incontinence diaper can be produced in an economic manner and undesired edges are avoided.

In this severing process, the coherent offcut formed by the rear side portion, the main part and the front side portion in each case has to be removed from the process. This is carried out in an advantageous manner by the method described in DE102008056220. In this regard, full and express reference is made to the disclosure content of DE102008056220.

It would also be conceivable to carry out the contouring of the side portions before the side portions are attached to the main part, for example on a still endless side-portion web.

According to a further concept of the invention, a distance C of the closure means, in particular of the closure means close to the leg opening, from the lower edge, facing the crotch, of the rear side portions is at most 8.0 cm, preferably at most 7.0 cm, particularly preferably at most 6.5 cm and very particularly at least 0.5 cm and more particularly at least 1.0 cm.

The basis weight of the material forming the rear side portions should preferably be 14-40 g/m², particularly 16-30 g/m² and very particularly 17-28 g/m².

The tear propagation resistance of the material forming the rear side portions, measured and determined as average force Fm, as described in more detail below, is preferably at least 5.0 N, particularly preferably at least 6.0 N and very particularly preferably at least 6.5 N, but preferably at most 10.0 N.

The tear propagation resistance of the material forming the rear side portions, measured and determined as average of the force peaks Fm.sp, as described in more detail below, is preferably at least 5.5 N, particularly preferably at least 6.0 N, very particularly preferably at least 6.5 N and particularly at least 7.0 N, but preferably at most 12.0 N.

The tear propagation resistance of the material forming the rear side portions, measured and determined as maximum peak force Fsp, as described in more detail below, is preferably at least 5.5 N, particularly preferably at least 6.0 N, very particularly preferably at least 6.5 N and particularly at least 7.0 N, but preferably at most 12 N.

In a further development of the invention, it has been found to be advantageous also to provide the front side portions with a basis weight as described above for the rear side portions. Preferably, the front side portions furthermore also have a tear propagation resistance Fm and/or Fm.sp and/or Fsp as described for the rear side portions.

The front side portions can per se have any desired shape, in particular a rectangular shape. However, the front side portions preferably likewise have contouring, and in particular a transverse edge, facing the crotch, of the front side portions can have a concave and/or a convex portion.

According to a preferred embodiment of the invention, an inner edge and an outer edge of the front and/or rear side portions extend parallel to one another. More preferably, the inner and/or outer edge extend(s) at least in portions, preferably entirely, that is to say along their entire extent, parallel to a longitudinal direction of the disposable incontinence diaper. Preferably, the inner edge has a greater extent D in the longitudinal direction than the outer edge.

It has further been found to be advantageous for the front and/or rear side portions to consist of a nonwoven material or to comprise a nonwoven material. In particular all nonwoven materials which contain at least one formulation component based on a thermoplastic polymer are suitable. The nonwovens may contain fibers of PE, PP, PET, rayon, cellulose, PA and mixtures of these fibers. Bicomponent and multicomponent fibers are also conceivable and advantageous. In particular, carded nonwovens, water-jet needled nonwovens, spunbonded (S) nonwovens, meltblown (M) nonwovens, SMS nonwovens, SMMS nonwovens or else laminates of one or more of these kinds of nonwoven, wherein S stands for spunbonded nonwoven layers and M for meltblown nonwoven layers, are advantageous. It is furthermore conceivable and advantageous to form the front and/or rear side portions from a nonwoven-film laminate. In such a case, the film component would come to lie on the outside and the nonwoven component on the inside in order to ensure that a soft surface faces the body. As a development of this concept of the invention, it is advantageous to form the front and/or rear side portions from a nonwoven-film-nonwoven laminate, in which a film component is arranged in the manner of a sandwich between two nonwoven components.

Furthermore, it has been found to be advantageous that, laterally next to the longitudinal edges of the absorbent pad, first elastic elements having a component in the longitudinal direction are attached to the main part. These elastic elements can extend exactly in the longitudinal direction, that is to say in a straight line, or, particularly advantageously, also be provided such that they follow a certain contouring along the leg openings. In such a case, the elastic elements follow a curved path along the leg opening. In a particular development of this concept of the invention, it is provided that the elastic elements do not extend into the side portions, but are limited to being positioned within the main part. Furthermore, second elastic elements that are made to extend in the first longitudinal direction and in particular are in the form of what are known as upright cuff elements, which are known per se, for example including from EP0263720A1, may be attached to the web of the main part of the diaper. These preferably upright second elastic elements flank to a certain extent a center of the main part of the diaper or absorbent pad; they may be provided in the region of the edges of the absorbent pad, within the edges of the absorbent pad or outside the edges of the absorbent pad. They form a lateral run-out guard for the disposable incontinence diaper.

In a development of the invention, it is provided that, in order to secure the disposable incontinence diaper as intended on the body of a person, the closure means can be secured in a detachable manner at least in regions both to the outer side of the main part and also to the outer side of the front side portions, with the retaining forces between the closure means and the outer side of the front side portions preferably being greater than the retaining forces between the closure means and the outer side of the main part. This makes the user in most cases secure the closure means to the front side portions. The retaining forces determined as over-the-stomach retaining forces between the closure means, which have in particular mechanical closure aids, and the outer side of the main part are preferably 20-57 N/25 mm, particularly 25-50 N/25 mm. Furthermore, the over-the-stomach retaining forces between the closure means and the outer side of the side portions in the front region are preferably 58-90 N/25 mm, particularly 60-80 N/25 mm. Furthermore, it has been found to be advantageous for the over-the-stomach retaining forces between the closure means and the outer side of the rear side portions to be lower than the over-the-stomach retaining forces between the closure means and the outer side of the front side portions. This, too, makes the user in most cases secure the closure means to the front side portions. In the context of the present invention, the over-the-stomach retaining forces were determined by the test method described in WO2008049546A1.

Advantageously, the rear side portions have at least one further closure means which is remote from the leg opening and is provided with closure aids.

The outer side of the main part of the disposable incontinence diaper is preferably formed by a nonwoven material, at least in regions, but particularly over the entire surface area. This gives the disposable incontinence diaper a “textile-like” impression. In such a case, it is advantageous to form the back sheet of the main part from a nonwoven-film laminate, with the nonwoven layer coming to lie on the outside and the film layer on the inside directed toward the absorbent pad, so that the nonwoven layer forms the outer side of the main part. This both ensures the liquid-impermeability of the main part and ensures the skin-friendly nature of the diaper. The film layer of this nonwoven-film laminate is then formed preferably from a one- or multi-layer liquid-impermeable, but preferably nevertheless breathable, film, with the breathability of the front and/or the rear side portions preferably being greater than the breathability of the nonwoven-film laminate forming the back sheet of the disposable incontinence diaper.

Advantageously, the rear side portions differ from the front side portions with regard to at least one, particularly at least two, more particularly at least three, and more particularly at least four, of their primary properties selected from the group comprising type of material, basis weight, breathability, density, stretchability, closure force, surface extent, thickness, and color. In this regard, express reference is made to the disclosure content of WO2009/015746.

In a development of the invention, it has been found to be advantageous for the length of the front and/or rear side portions, that is to say their maximum extent in the longitudinal direction of the diaper, to be at least 15 cm, particularly at least 20 cm, more particularly at least 25 cm, more particularly at least 27 cm and more particularly at most 45 cm. Advantageously, the overall length of the disposable incontinence diaper is 60-120 cm, particularly 65-115 cm and more particularly 70-110 cm. In a development of the invention, it has been found to be advantageous for the width of the front and/or rear side portions, that is to say the maximum extent of the side portions beyond the lateral edge of the main part of the diaper, to be 10-40 cm, particularly 12-35 cm, more particularly 13-30 cm. Preferably, the front side portions are the same width as the rear side portions.

In the drawing:

FIG. 1 shows a schematic illustration of a plan view, not to scale, of a disposable incontinence diaper having side portions attached on both sides;

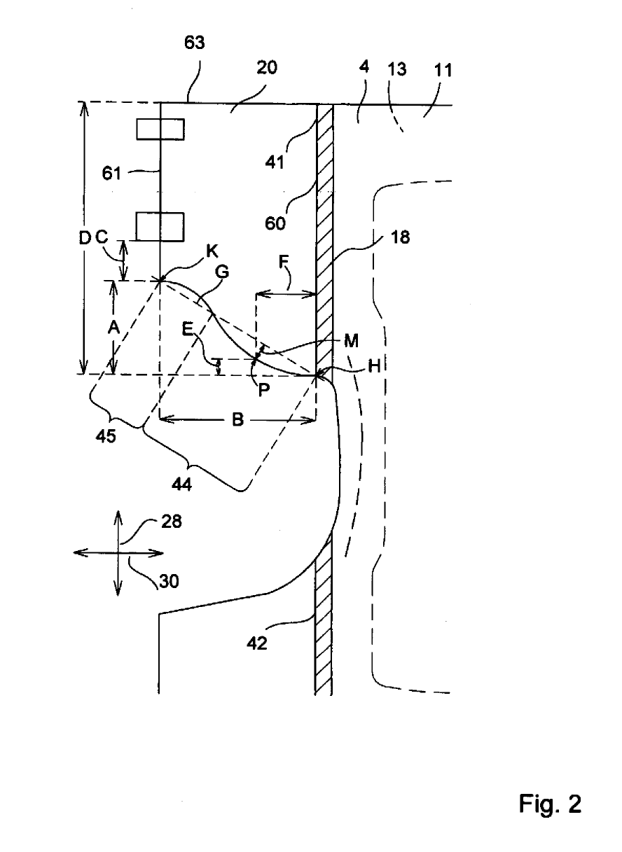

FIG. 2 shows an enlarged partial view of the disposable incontinence diaper according to FIG. 1;

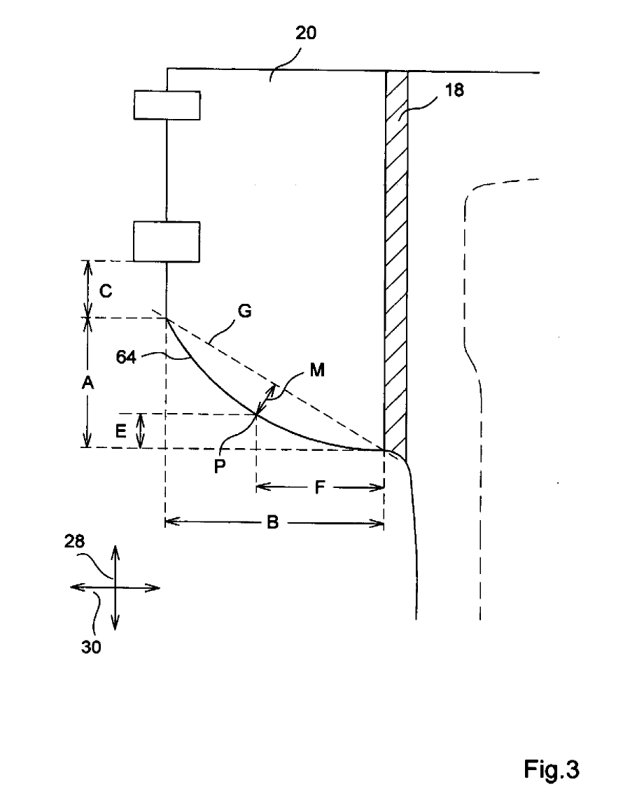

FIG. 3 shows by way of example a further side-portion geometry of a disposable incontinence diaper according to the invention (not to scale);

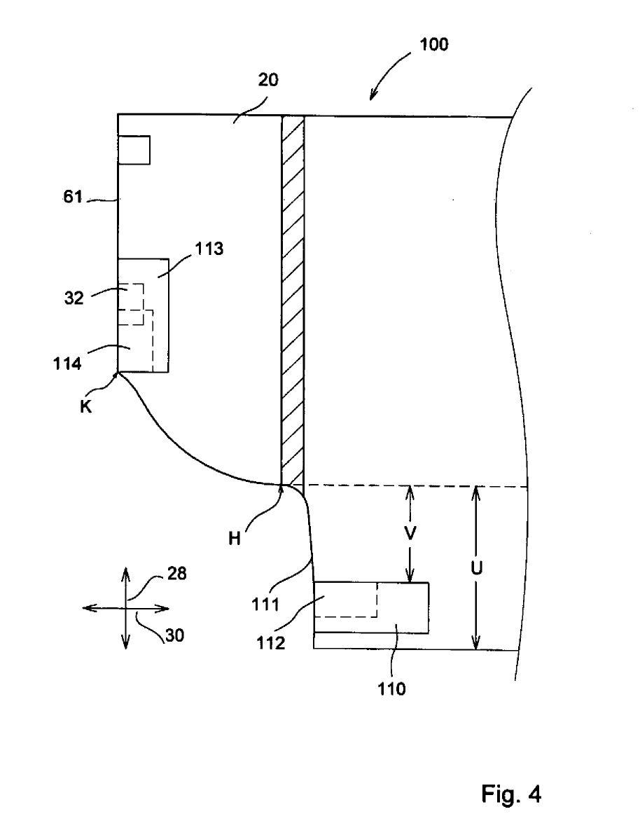

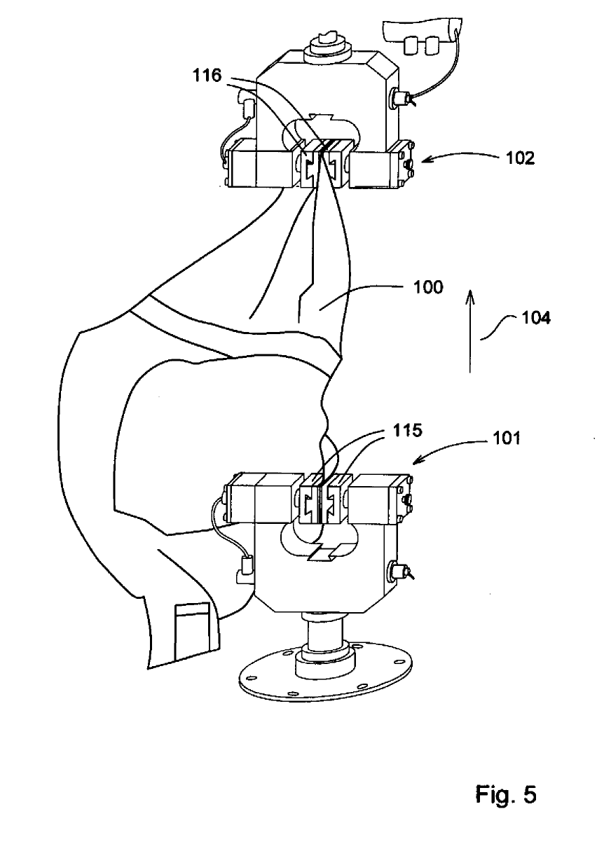

FIGS. 4 and 5 show an illustration of the testing of the side-portion tear strength.

FIG. 1 schematically shows, not to scale, a plan view of the inner side, that is to say the side facing the body, of a first embodiment (example 1) of the disposable absorbent incontinence diaper 2 of the open type in the just-unfolded state. The disposable incontinence diaper 2 comprises a main part 4 having a front region 6, a rear region 8 and a crotch region 10 located in between in the longitudinal direction 28. Also indicated is an absorbent pad 12, which is usually arranged between chassis-forming materials of the main part 4, that is to say particularly between a liquid-permeable top sheet 11, which is formed from a nonwoven material, and a substantially liquid-impermeable back sheet 13, which is formed from a film material, of the main part 4. The back sheet 13 can also be formed from a liquid-impermeable nonwoven material or from a nonwoven-film laminate, with the nonwoven layer then coming to lie on the outside and the film layer on the inside in a manner directed toward the absorbent pad. This gives the disposable incontinence diaper 2 a “textile-like” impression. Laterally next to the longitudinal edges of the absorbent pad 12, first elastic elements 80 are attached to the main part 4, between the top sheet 11 and the back sheet 13. The elastic elements 80 extend substantially in the longitudinal direction 28, that is to say with a substantial component in the longitudinal direction, wherein in the illustrated case they follow a curved path along the leg-opening region portion to be assigned to the crotch region 10. The disposable incontinence diaper 2 furthermore comprises front side portions 22 and rear side portions 20, which are attached to the main part 4 on both sides as four separate nonwoven components. The lower edge 64 of the rear side portions 20 has a convex portion 44 and a concave portion 45, while the front side portions 22 have a continuously concave leg-opening contour. As is shown in an enlarged illustration, not to scale, of a partial view of FIG. 1 (FIG. 2), the side portions 20, 22 are connected in a nondetachable manner in an overlapping region 18, which is illustrated in a hatched manner, to chassis-forming materials of the main part 4, that is to say, for example, to the back sheet 13 and/or the top sheet 11. The side portions 20, 22 extend in the transverse direction 30 beyond the front and rear lateral longitudinal edges 42, 41 of the main part.

The front and rear lateral longitudinal edges 42, 41 of the main part are understood in the context of the present invention to mean those longitudinal edge regions of the main part to which the side portions are attached and beyond which said side portions extend.

The longitudinal extent of the front and rear lateral edges 42, 41 of the main part thus also define the longitudinal extent of the front region 6 and of the rear region 8 of the disposable incontinence diaper 2.

Hereby, it is also made clear that the expressions “lateral edge” and “lateral longitudinal edge” are used as synonyms in the preceding and following text.

The side portions 20, 22 are designed and intended to be connected to one another in the put-on state of the disposable incontinence diaper 2, in order to form a hip region of the sanitary article that is continuous in the circumferential direction. In this case, the side portions 20, 22 provided on one side of the main part 4 are in each case connected together. To this end, mechanical closure means 32 close to the leg opening and closure means 33 remote from the leg opening, particularly having mechanical closure aids such as burr hooks, are provided on the rear side portions 20 and can be secured in a detachable manner to the outer side of the front and rear side portions 20, 22. Preferably, the closure means can also be secured in a detachable manner to the outer side of the main part such that the incontinence article can be matched in a very variable manner to the anatomical conditions of the wearer. Both the front side portions 22 and the rear side portions 20 are formed from a nonwoven material, in the illustrated case from a PP spunbonded nonwoven, Pegatex S, manufacturer: Pegas a.s., Primetická 86, 66904 Znojmo, CZ. The basis weight of the nonwoven material of the front side portions is 30 g/m². The fiber thickness of the fibers forming the nonwoven material is 2 dtex. The over-the-stomach retaining forces between the closure means 32 and the outer side of the front side portions 22 are preferably at least 58 N/25 mm.

The basis weight of the nonwoven material of the rear side portions 20 in the illustrated case is 27 g/m².

The tear propagation resistance of the nonwoven material of the rear side portions, measured in the longitudinal direction 28, is:

### Fm: 7.0 N

### Fm.sp: 7.2 N

### Fsp: 9.8 N.

The over-the-stomach retaining forces between the closure means 32, 33 and the outer side of the rear side portions 20 are lower than the over-the-stomach retaining forces between the closure means 32, 33 and the outer side of the front side portions 22.

As can be seen from FIG. 1, the rear side portions 20 have a larger surface extent than the front side portions 22.

The front and rear side portions thus differ in at least three of their primary properties, namely the basis weight, the closure force and the surface extent.

The difference in closure force between the front and rear side portions makes the user secure the closure means 32, 33 preferably to the front side portions 22, this being beneficial to the fit of the diaper. As can further be gathered from FIG. 1, the leg-opening regions 50 are formed by front and rear side portions 20, 22, which are formed in a curved manner toward the crotch region and form the side-portion leg-opening regions, and also by the hourglass-shaped contouring of the main part 4. An hourglass-shaped contouring of the main part is understood here to mean any shape of the narrowing in the main part 4 in the crotch region 10, that is to say any curved, and also any non-curved or not exclusively curved shape, in which case the crotch region 10 of the main part 4 has a shorter extent in the transverse direction 30 than the front region 6 and/or the rear region 8 of the main part.

The stability of the leg opening 50 is formed by a respective single cut, i.e. one on each side, said cut taking in both the side portions 20, 22 and the main part 4 and in the process being guided continuously through lateral-edge and main-part material to be separated. The leg opening 50 thus comprises a front side-portion leg-opening region 52 and a rear side-portion leg-opening region 51 and the main-part leg-opening region 53 (FIG. 2).

FIG. 2 also illustrates the positioning of the closure means 32 close to the leg at the distance C and the determination of the length A and the width B of the lower edge 64 of a rear side portion 20. The rear side portion 20 is bounded by an inner edge 60 which corresponds to the rear lateral edge 41 of the main part, by an outer edge 61, and also by an upper edge 63 and a lower edge 64 which forms the contour of the side-portion leg-opening region. The upper edge 63 and the lower edge 64 connect the inner edge 60 to the outer edge 61. The inner edge 60 and the outer edge 61 preferably extend parallel to one another, more preferably, the inner edge 60 and/or outer edge 61 extend at least in portions parallel to a longitudinal direction 28 of the disposable incontinence diaper. The inner edge 60 preferably has a greater extent D in the longitudinal direction 28 than the outer edge 61.

In the context of the present invention, the length A is defined as the greatest extent of the lower edge 64 in the longitudinal direction 28. The width B is defined as the greatest extent of the lower edge 64 in the transverse direction 30. The distance C is defined as the shortest distance, to be determined in the longitudinal direction 28, between a closure means 32 close to the leg opening and the lower edge 64.

The further variables are determined as follows in the context of the present invention:

In order to determine the extreme point P, a straight line G is drawn through the inner end point H and the outer end point K of the lower edge 64. An outwardly curved, i.e. convex, portion 44 of the lower edge 64 of the rear side portions is defined with respect to this straight line G. In the illustrated case, the lower edge 64 also has an inwardly curved, concave, portion 45 with respect to the straight line G. The extreme point P is that point on the lower edge 64 within the convex portion which has a maximum distance M between the straight line G and the lower edge 64, wherein the distance should be measured perpendicular to the straight line G. Should it not be possible to determine the extreme point P clearly by this method, for instance because the lower edge 64 has more than one convex portion or because the convex portion has more than one point having a maximum distance from the straight line G, then that point is chosen which is located closest to the inner edge 60 in the transverse direction 30.

The longitudinal extent E of the extreme point P is the distance, measured in the longitudinal direction 28, of the extreme point P from an imaginary line which extends through the inner end point H of the rear side portion 20 parallel to the transverse direction 30.

The transverse extent F of the extreme point P is the distance, measured in the transverse direction 30, of the extreme point P from an imaginary line which extends through the inner end point H of the rear side portion 20 parallel to the longitudinal direction 28.

In the illustrated first embodiment (example 1), the ratio of the length A of the lower edge of a rear side portion to the width B of the lower edge of a rear side portion is 0.63. The ratio of the longitudinal extent E of the extreme point P of the convex portion to the transverse extent F thereof is 0.27.

FIG. 3 shows an alternative side-portion geometry according to the invention (example 2), wherein the lower edge 64 of a rear side portion 20 is curved outwardly in its entirety with respect to the straight line G, i.e. the lower edge 64 consists of a single convex portion. Once the extreme point P has been determined as the point which has a maximum distance M from the straight line G, the further variables can be determined in an analogous manner, as described above. The ratio of the length A of the lower edge of a rear side portion to the width B of the lower edge of a rear side portion is 0.64 here. The ratio of the longitudinal extent E of the extreme point P of the convex portion to the transverse extent F thereof is 0.40. The distance C of the lower edge 64 from the closure means close to the leg opening is 6 cm.

The following table summarizes the variables A-F and also the ratios A:B, E:F, E:A and F:B:

| | Example 1 | Example 2 |
|---|---|---|
| A in mm | 8.3 | 8.4 |
| B in mm | 13.2 | 13.2 |
| C in mm | 3.0 | 6.0 |
| D in mm | 30.5 | 35.0 |
| E in mm | 1.1 | 2.0 |
| F in mm | 4.1 | 5.0 |
| A:B | 0.63 | 0.64 |
| E:F | 0.27 | 0.40 |
| E:A | 0.13 | 0.24 |
| F:B | 0.31 | 0.38 |

The work, measured in mNm up until breakage of the materials was 2047 mNm in example 1, 2034 mNm in example 2 and 1901 mNm in a comparative example not according to the invention. As comparative example, an otherwise structurally identical incontinence article, in which the lower edge of the rear side portions has an exclusively concave portion, as illustrated in FIGS. 2a and 2b in DE102009015041, was tested.

In the context of the present invention, the side-portion tear strengths are measured by means of the test method explained further below.

In the context of the present invention, the tear propagation resistances are determined as tear propagation strength by the test method specified in DIN EN ISO 13937-2. As a deviation from this, the sample length is 150 mm. The central incision has a depth of 50 mm. The speed of deformation is set to 200 mm/min. The evaluation takes place by means of an electronic device. As a deviation from DIN EN ISO 13937-2, a peak value to be evaluated is characterized by an increase or drop in force of at least 0.2 N. Thus, upper and lower force peaks are taken into account when the Fm.sp is being determined. Besides the arithmetic average value of the force peaks Fm.sp, the maximum value of all the upper force peaks Fsp of each specimen and the average force Fm calculated over the entire force characteristic curve to be evaluated are also determined.

The present invention has thus been successful in providing for a disposable incontinence diaper having front and rear side portions attached to the main part on both sides, said disposable incontinence diaper having a further improved side-portion tear strength during use and also taking sufficient account of the comfort of putting on and wearing the disposable incontinence diaper.

### Side-Portion Tear Strength Test Method

The test method is described with reference to FIGS. 4 and 5. The incontinence articles to be tested are first of all stored for 12 h under standard conditions at 23° C. and 50% relative humidity.

A disposable incontinence diaper is separated (cut or punched) through the entire crotch region, at a distance U of about 150 mm below the lower end point H, with the main part being damaged in the transverse direction 30. A self-adhesive first textile tape 110 about 50 mm wide and about 240 mm long is fixed to the specimen 100 obtained in this way at a distance V of 100 mm below the inner end point H on both sides, that is to say on the surface forming the inner side of the main part and the surface forming the outer side of the main part, to reinforce the material, said textile tape to this end being folded over at the longitudinal edge 111 of the corresponding crotch-region portion, such that the tape covers an area of about 50 mm (in the longitudinal direction 28)×120 mm (in the transverse direction 30) on both sides. The first textile tape and also the second textile tape described below serve merely to fix the corresponding portions securely in the clamps of the tensile testing instrument, as described in more detail below. It is suitable to obtain Omnitape® from Paul Hartmann AG, Heidenheim, Germany, for example. The first position 112 of the lower, fixed clamp 101 of the tensile testing instrument is marked on the first tape 110, flush with the longitudinal edge 111 and the upper edge of the first tape 110 (that is to say at a distance V of 100 mm below the rear side portion), with an extent of 60 mm in the transverse direction 30 and 30 mm in the longitudinal direction 28. Immediately above the outer end point K, an approximately 50 mm wide self-adhesive second textile tape 113 is fixed likewise on both sides to reinforce the material, such that the tape 113 covers an area of about 50 mm (in the transverse direction 30 )×120 mm (in the longitudinal direction 28) on both sides. A closure means 32 close to the leg opening can in this case, depending on the arrangement of the closure means, be overlaid by the second tape 113. The second position 114 of the upper clamp 102 of the tensile testing instrument is marked on the second tape 113, flush with the outer edge 61 and the lower edge of the second tape 113, with an extent of 30 mm in the transverse direction and 60 mm in the longitudinal direction.

The specimen 100 is then clamped into a tensile testing instrument according to DIN EN ISO 75001, accuracy class 1, as follows:

The lower, fixed clamp 101 is fastened at the previously marked first position 112 by means of the two clamping jaws 115 having a width of 60 mm and a clamping depth of 30 mm, in that the portion in question is clamped firmly between the two clamping jaws 115 of the fixed clamp 101. The movable clamp 102, arranged perpendicularly above the fixed clamp 101, is fastened at the previously marked second position 114 by means of the two clamping jaws 116 having a width of 60 mm and a clamping depth of 30 mm, in that the portion in question is clamped firmly between the two clamping jaws 116 of the movable clamp 102. A tensile test is carried out by way of controlled movements of the movable clamp 102 in the direction of the arrow 104. The test speed at which the movable clamp 102 is moved mechanically vertically away from the lower clamp is 500 mm/min, with an initial force of 0.5 N (Newton) being first of all applied. Overall, a minimum number of n=5 tests are intended to be carried out. For evaluation purposes, the work in [mNm] applied up until the maximum force (breakage of the materials) in each tensile test is determined via integration of the stress-strain curve and subsequently the arithmetic mean of the individual values is calculated.

1.-12. (canceled)

13. A disposable absorbent incontinence diaper of the open type, comprising:
a main part, said main part comprising
an absorbent pad;
a front region having front lateral longitudinal edges;
a rear region, having rear lateral longitudinal edges;
front side portions attached to respective sides of the front region;
rear side portions attached to respective sides of the rear region and having a lower curved edge for forming rear side portion leg openings, said front and rear side portions extending in a transverse direction beyond the rear and front lateral longitudinal edges, wherein the rear side portions have first closure means having closure aids and being arranged proximal to a leg opening of the incontinence diaper, said closure means being detachably securable at least on an outer side of the front side portions, for connecting the front region and the rear region to one another; and
a crotch region located between the front region and the rear region in a longitudinal direction and being arranged between the legs of a user, wherein a ratio of a length of the lower edge to a width of the lower edge is 0.40-0.90 and a ratio of a longitudinal extent of an extreme point of a convex portion of the lower edge to a transverse extent of the extreme point is 0.15-0.80.

14. The disposable absorbent incontinence diaper of claim 1, wherein a ratio of the transverse extent of the extreme point to the width of the lower edge of the rear side portion is 0.20-0.60, particularly 0.30-0.50.

15. The disposable absorbent incontinence diaper of claim 1, wherein a ratio of the longitudinal extent of the extreme point to the length of the lower edge of the rear side portion is 0.10-0.40, particularly 0.10-0.30.

16. The disposable absorbent incontinence diaper of claim 1, wherein the ratio of the longitudinal extent of the extreme point to the transverse extent of the extreme point is 0.20-0.60, particularly 0.20-0.50.

17. The disposable absorbent incontinence diaper of claim 1, wherein the rear side portions are formed by a material having a tear propagation resistance Fm of at least 4.0 N in the longitudinal direction.

18. The disposable absorbent incontinence diaper of claim 1, wherein the ratio of the length of the lower edge of the rear side portion to the width of the lower edge of the rear side portion is 0.50-0.80, particularly 0.55-0.75.

19. The disposable absorbent incontinence diaper of claim 1, wherein the closure means are arranged at a distance from the lower edge of the rear side portions of at most 8.0 cm, particularly at most 7.0 cm, more particularly at most 6.5 cm and more particularly at least 0.5 cm.

20. The disposable absorbent incontinence diaper of claim 1, wherein the rear side portions have at least one further said closure means arranged distal to the leg opening.

21. The disposable absorbent incontinence diaper of claim 1, wherein the rear side portions are formed by a material having a tear propagation resistance Fm of at least 5.0 N, particularly at least 6.0N, particularly at least 6.5 N and more particularly at least 10.0 N in the longitudinal direction.

22. The disposable absorbent incontinence diaper of claim 1, wherein the rear side portions are formed by a material having a tear propagation resistance Fm.sp of at least 5.5 N, particularly at least 6.0 N, particularly at least 6.5 N and more particularly at least 7.0 N and more particularly at most 12 N in the longitudinal direction.

23. The disposable absorbent incontinence diaper of claim 1, wherein the rear side portions are formed by a material having a tear propagation resistance Fsp of at least 5.5 N, particularly at least 6.0 N, particularly at least 6.5 N and more particularly at least 7.0 N and more particularly at most 12.0 N in the longitudinal direction (28).

24. The disposable absorbent incontinence diaper of claim 1, wherein the rear and front side portions are formed from a nonwoven material or comprise a nonwoven material.

25. The disposable absorbent incontinence diaper of claim 1, wherein the rear and front side portions extend beyond the rear and front lateral longitudinal edges of the main part by 10-40 cm, particularly 12-35 cm, more particularly 13-30 cm.

26. The disposable absorbent incontinence diaper of claim 1, wherein a length of the rear side portions in the longitudinal direction is at least 15 cm, particularly at least 20 cm, more particularly at least 25 cm and more particularly at least 27 cm and more particularly at most 45 cm.
